(19) United States
(12) Patent Application Publication
(10) Pub. No.: US 2005/0165376 A1
(43) Pub. Date: Jul. 28, 2005

(54) Designing dry and porous absorbent composites containing super-absorbent polymers

(76) Inventors: Fredric L Buchholz, Midland, MI (US); David S. Allan, Midland, MI (US); Joseph L. Weir, Midland, MI (US)

Correspondence Address: THE DOW CHEMICAL COMPANY;INTELLECTUAL PROPERTY SECTION, P. O. BOX 1967, MIDLAND, MI, 48641-1967, US

(21) Appl. No.: 10/506,652

(22) PCT Filed: Feb. 19, 2003
(86) PCT No.: PCT/US03/04729

Related U.S. Application Data
(60) Provisional application No. 60/366,593, filed on Mar. 21, 2002.

(51) Int. Cl. A61F 13/15; A61F 13/20

(52) U.S. Cl. 604/385.01

(57) ABSTRACT

Super-absorbent polymer composites and a method for designing the composites are presented. A target weight of aqueous liquid to be absorbed is used in conjunction with a dryness quality value and, optionally, a porosity quality value to define types and masses of both super-absorbent polymer and a substruction meshwork which are intermixed to provide a composite which optimally minimizes free liquid and provides sustained tactile dryness after the targeted weight of aqueous liquid has been absorbed. In one form of the invention, the super-absorbent polymer and/or absorption composite derive from the use of computer-implemented determination of the absorption design-instance parameters.

This invention provides a method and apparatus for designing absorbent composites containing super-absorbent polymers that can absorb relatively large quantities of aqueous liquids.

Absorbent composite materials having super-absorbent polymers that can absorb large amounts of aqueous liquids, such as water or body fluids, have many applications in disposable absorbent articles such as baby diapers, feminine hygiene napkins, and incontinent pads. Preferably, the absorbent composites absorb and retain large amounts of liquids under moderate pressure. For example, in order to prevent leaks onto clothing and excessive skin wetness, a baby diaper must absorb and retain urine under a variety of applied pressures, such as those of body forces applied by (a) a sitting or reclining infant and (b) gravitational force. In addition, a baby diaper must also absorb a total amount of liquid applied in several instances spaced in time; as can be appreciated, absorption and retention in the partially wet or partially saturated condition is, therefore, also important.

The absorption and retention abilities of super-absorbent polymers conventionally are measured by standardized tests such as the centrifuged retention capacity (CRC) test (European Disposables and Nonwovens Association, Recommended Test Procedure No. 441.1-99) and the absorbency under load performance under pressure) test (European Disposables and Nonwovens Association, Recommended Test Procedure No. 442.1-99). The European Disposables and Nonwovens Association, Recommended Test Procedure No. 441.1-99 is also alternatively referenced as either the “teabag” test or the Centrifuge Retention Capacity test (CRC) and it's results define the Centrifuge Retention Capacity (or CRC) for a particular superabsorbent. These and other such tests are described in references such as F. L. Buchholz and A. T. Graham, editors., Modern Super-absorbent Polymer Technology, Wiley-VCH, New York, 1998, especially Chapter 4. The absorption and retention abilities of absorbent composites having super-absorbent polymers are conventionally measured by standardized tests such as the Saturated Retention Capacity (Kellenberger, et al., EP-443,627-A2, page 12) or the equilibrium demand-absorbency test (Goldman, et al., EP-304,319-B1, page 10). In addition, the wetness of the absorbent composites or their propensity to leak conventionally is measured by methods such as the Rewet test (sometimes referred to as i test) as described in K. T. Hodgson, TAPPI Journal, August 1991, pages 205-212. Current practice in the super-absorbent products industry is (a) to primarily define the practical absorbency of a super-absorbent polymer in terms of the aforementioned Centrifuge Retention Capacity test (CRC) and (b) to subsequently define the practical absorbency (theoretically calculated as a mass fraction-weighted absorbency based on the absorbencies of the individual components and the respective mass fractions in the composite) of any composites that contain the superabsorbent polymer in terms of the saturated retention capacity or the equilibrium demand-absorbency of the composite. An example of such a determination is described in Bewick-Sonntag, U.S. Pat. No. 5,836,929 column 11 lines 6-27. All of these conventional methods use either immersion of the sample in a large excess of the liquid or the provision of a reservoir of liquid of excess capacity in contact with either the super-absorbent polymer or the composite comprising super-absorbent polymer.

As the super-absorbent material tightly binds the liquid delivered to the composite in which it is disposed, the effectual absorption and retention of the liquid provides for a tactile dryness attribute in the composite as a whole; in this regard, the composite is not “dry” insofar as its super-absorbent has become laden with liquid, but the composite is reasonably “dry to the touch” (tactily dry) insofar as the liquid is held within the composite and a non-composite surface (that is, skin) in contact with the outer surface of the composite will not provide a preferential hydrophilic solid phase for promoting mass transfer of the liquid out of the composite; in this regard, the skin of a baby using a composite will generally continue to be and feel reasonably “dry” respective to the amount of liquid absorbed into the composite.

The characteristics of the composite change during use (during the process of liquid absorption and retention) as the composite changes from an initial dry and compact state to a wetted and swollen state; the use process therefore generates a composite with differentially-modified performance characteristics through its use cycle. The permeability, or saline flow conductivity, of absorbent composites is an important parameter in their design and application. The permeability of porous media is fundamentally related to the porosity of the porous media, with permeability generally increasing with increasing porosity. Current practice in the hygiene products industry derives from the belief (a) that the porosity or liquid permeability of composites that contain super-absorbent polymer correlates to the liquid permeability of a granular bed of the swollen super-absorbent polymer and (b) that the design of the composite is based on this correlation.

However, the dynamic modification of performance properties during the use cycle creates a challenge in designing a super-absorbent composite of full effectiveness; and the use, for composite design, of performance parameters defined at the final or terminal state of use do not fully and efficiently anticipate the set of considerations which affect a composite during its use cycle. In this regard, the needs for quality performance and cost minimization establish a need for a design technique which predictably defines a composite enabling a tight binding of all the liquid delivered to the composite while providing that the wetted and swollen composite possesses optimal porosity (and therefore improved liquid permeability) for subsequent additions of liquid to the composite. The present invention fulfills this need.

The invention is for a super-absorbent polymer cumulation (with the polymer having an affiliated centrifuge retention capacity value and where the term “cumulation” references a collective instance of all super-absorbent material in either unified or dispersed form which is relevant to absorbing a targeted weight of liquid) for absorbing a targeted weight of aqueous liquid, where the super-absorbent polymer cumulation has a super-absorbent mass between a 1.18 and a 2.22 multiple of

$$K = \frac{{m}_{liq}}{CRC}$$

where
   m_{liq} is a value denoting the targeted weight of liquid, and
   CRC is the centrifuge capacity value has units of mass of liquid per mass of dry super-absorbent polymer
      so that a sufficiently minimal amount of super-absorbent polymer cumulation is provided for effectively minimizing free aqueous liquid within the super-absorbent polymer cumulation after the targeted weight of aqueous liquid has been absorbed such that the super-absorbent polymer cumulation with the targeted weight of absorbed aqueous liquid provides sustained tactile dryness. K is denoted as a absorption design-instance parameter in the above equation.

When the super-absorbent polymer cumulation is dispersed throughout a permeable substruction meshwork (with “substruction” referencing the underlying supportive nature of the meshwork in the absorbent medium fabricated from the polymer and the meshwork), the invention is also for an absorbent medium for absorbing a targeted weight of aqueous liquid where the medium incorporates super-absorbent polymer (having an affiliated centrifuge retention capacity value) dispersed throughout the permeable substruction meshwork, the permeable substruction meshwork has a mass of intertwined stranding (the stranding has an affiliated absorption capacity value), and the super-absorbent polymer cumulation has a super-absorbent mass between a 1.18 and a 2.22 multiple of an absorption design-instance parameter derived from the aqueous liquid weight, the absorption capacity value, the mass of stranding, and the centrifuge retention capacity value according to

$$K = {m}_{liq} ( \frac{{m}_{liq} - {C}_{stranding} {m}_{stranding}}{{CRC m}_{liq}} )$$

where
   m_{liq} is a value denoting the targeted weight of liquid,
   C_{stranding} is the absorption capacity value has units of mass of liquid per mass of dry stranding,
   CRC is the centrifuge capacity value has units of mass of liquid per mass of dry super-absorbent polymer,
   m_{stranding} is a value denoting the mass of stranding, and
   K is the absorption design-instance parameter;
      so that a sufficiently minimal amount of super-absorbent polymer is provided for effectively minimizing free aqueous liquid within the absorbent medium after the targeted weight of aqueous liquid has been absorbed such that the absorbent medium with the targeted weight of absorbed aqueous liquid provides sustained tactile dryness.

In another form, the invention is for a method of making an absorbent medium using the step of intermixing permeable substruction stranding (having an affiliated absorption capacity value) and a mass of super-absorbent polymer particles (having an affiliated centrifuge retention capacity value as determined below) into a meshwork for absorbing a predefined mass of liquid to a predefined dryness quality (denoted by a dryness quality value between 0.45 and 0.85 where 0.45 denotes an absorbent medium having a maximal dryness quality after absorption of the liquid mass and 0.85 an absorbent medium having a minimal dryness quality after absorption of the liquid mass) with the cumulative mass of all the stranding being

$${m}_{stranding} = \frac{{m}_{liq} - Φ ( CRC ) {m}_{sap}}{{C}_{stranding}}$$

where
m_{stranding} is a value denoting the cumulative mass of all the stranding,
m_{liq} is a value denoting the predefined mass of liquid to be absorbed,
Φ is the dryness quality value,
CRC is the centrifuge retention capacity value having units of mass of liquid per mass of dry super-absorbent polymer,
mₛₐₚ is a value denoting the cumulative mass of all the super-absorbent polymer particles, and
C_{stranding} is the absorption capacity value having units of mass of liquid per mass of dry stranding,
where the super-absorbent polymer particles and stranding are intermixed to further achieve a predefined porous quality (denoted by a porous quality value between 0.4 and 0.95 where 0.4 denotes an absorbent medium having a minimal porous quality after absorption of the liquid mass and 0.95 denotes an absorbent medium having a maximal porous quality after absorption of the liquid mass), and
where the centrifuge retention capacity value is determined according to

$$CRC = {F [ {( \frac{1}{{R}_{ϕ}} )}^{\frac{1}{{f}_{s}^{1.83} + 0.07}} - 1 ]}^{0.54} ,$$
where
F is 40.58 with units of mass of liquid per mass of dry super-absorbent polymer,
R_{φ} is the porous quality value, and
fₛ is the super-absorbent polymer mass fraction value according to

$${f}_{s} = \frac{{m}_{sap}}{{m}_{sap} + {m}_{stranding}} .$$

In one form of the invention, the super-absorbent polymer and/or absorption composite derive from the use of computer-implemented determination of the absorption design-instance parameter.

Further features and details of the present invention are appreciated from a consideration of the Detailed Description of the Preferred Embodiments of the invention and the accompanying Figures.

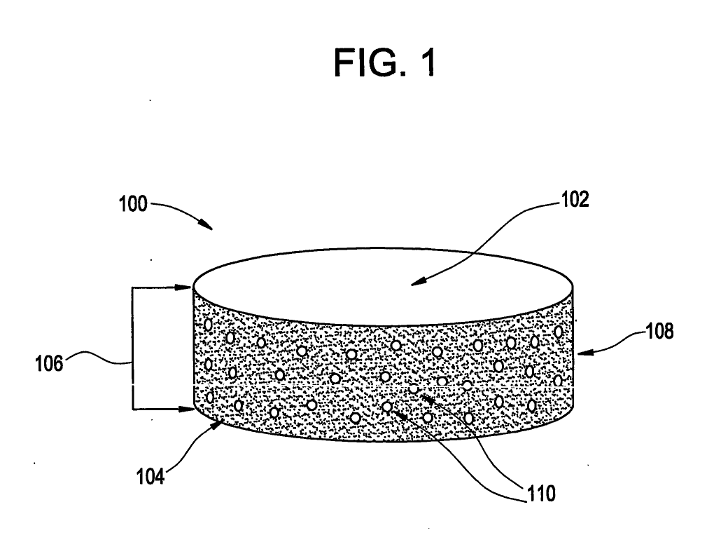

FIG. 1 presents an architectural model of an absorbent composite.

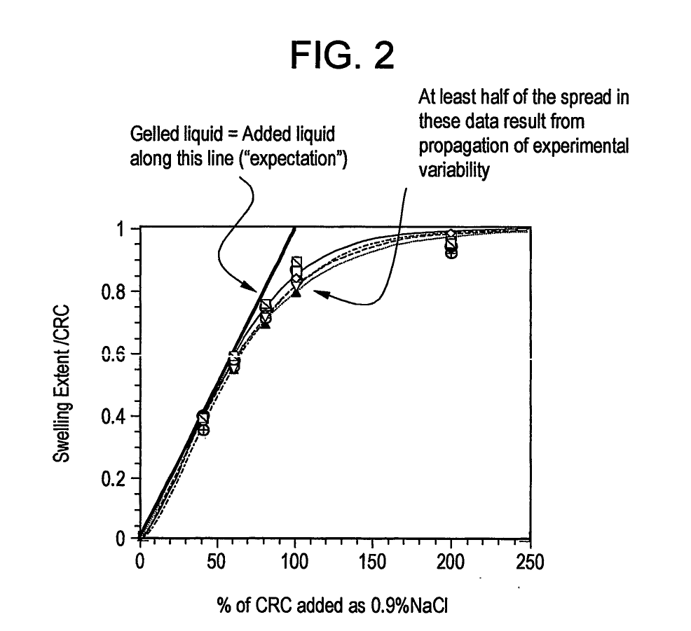

FIG. 2 presents graphical information respective to dryness quality in super-absorbent composites.

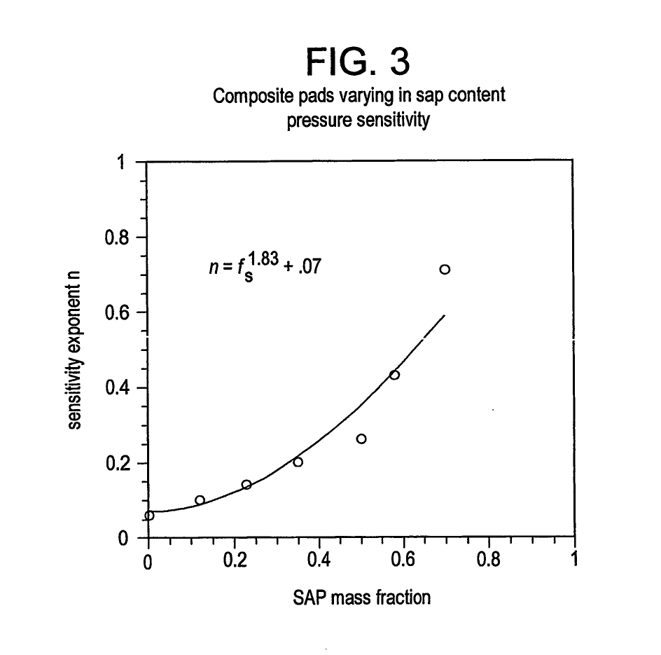

FIG. 3 presents graphical information respective to porosity considerations in super-absorbent composites.

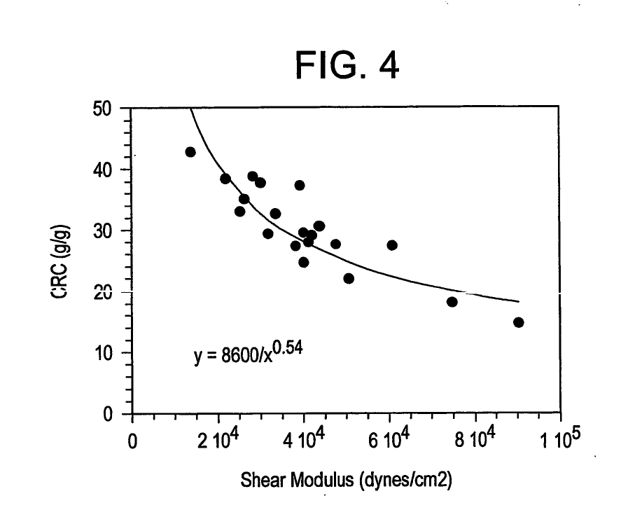

FIG. 4 presents graphical information respective to critical shear (elastic) moduli and corresponding swelling ratios in saturated super-absorbent composites.

In designing an absorbent composite, a number of considerations must be resolved into acceptable balance. A super-absorbent material (“super-absorbent”) must be identified and is defined herein in the context of a particular super-absorbent chemical composition in a particular physical form and in a particular overall quantity. If a permeable substruction meshwork is also planned for the composite, then this must also be defined in the context of a particular chemical composition or material type in a particular physical form and in a particular quantity. The composite also has an architectural form characterized by outside dimensions and also usually by internal sections or layers, with each section or layer having its particular dimensions and structural nature. One of these layers must be the absorbent medium, that section of the composite containing the super-absorbent material for tightly binding all the liquid delivered to the composite and maintaining the bound liquid from migrating to the tactile (touchable) outer surfaces of the composite; the absorbent medium optionally (but usually) includes a permeable substruction meshwork through which the super-absorbent material is dispersed, although it is to also be noted that the absorbent medium in one embodiment is a bed of super-absorbent particles without the benefit of a substruction meshwork. As a distinctive domain, the performance of the absorbent medium affords characterization in a modeled context, and the modeled context provides the basis for the particular features of the present invention as further described herein.

While the present invention must necessarily consider detail in each of the above considerations to provide a usable absorbent product, it is to be appreciated that two surprising experimental finds are key in the characterization of the absorbent medium. In this regard, the inventors have discovered in a first surprising find that, under the real conditions of use of absorbent products (such as, for example but without limitation, diapers), existing super-absorbent polymers do not absorb the amount of liquid as determined by the CRC method but rather absorb much less liquid than that predicted by the CRC method. The amount absorbed under realistic conditions (a limited amount, rather than an “infinite” supply or large excess of liquid) falls into the range of 45-85 percent of the CRC value according to the findings as presented herein. Therefore, while absorbent designs of current practice rely on an unmodified CRC value, those of the present invention rely on a specified fraction of the CRC value. This important difference allows the media and derived composites of the present invention to tightly bind all the liquid delivered to the composite, and thereby exhibit superior dryness compared to current absorbent composites. Details in this first surprising find are further described herein.

The inventors have further discovered an improved method of specifying the amount and properties of super-absorbent polymer in a composite such that the wetted and swollen composite has optimal porosity and therefore improved liquid permeability for subsequent additions of liquid to the composite. In this regard, a higher fraction of super-absorbent in gel form from addition of liquid to the composite results in decreased porosity and permeability. Respective to porosity, the inventors have made a second set of related surprising discoveries that, when the composite absorbs liquid, the volume change of the composite scales up by a factor much smaller than that of the mass change and that, under compressive pressure, the compressibility of the swollen composite is efficaciously modeled in terms of a straightforward scaling function of (a) the applied pressure and (b) the elastic modulus of the swollen gel, with the scaling exponent of the function depending on the mass fraction of super-absorbent polymer particles in the absorbent composite according to an essentially smooth function. The inventors have further discovered that, for an optimally porous composite, there exists a critical value of the CRC above which the composite will exhibit poor porosity in the swollen state. The importance of this is understood by reprising the Background statement of contrasting current practice in the hygiene products industry where (a) the porosity or liquid permeability of composites that contain super-absorbent polymer are correlated to the liquid permeability of a granular bed of the swollen super-absorbent polymer and (b) that the design of the composite is based on this correlation. Details in this second surprising find are further described herein.

Turning now to the super-absorbent materials for use in the preferred embodiments, super-absorbents are usually chemically referenced as super-absorbent polymer—a water-insoluble but swellable polymer generally capable of absorbing several times (preferably greater than 10 times) its mass of water or other aqueous liquids into its molecular structure., absorbent, polymeric compositions of the present invention are materials capable of absorbing large quantities of fluids (that is, liquids) such as water and/or body exudates (for example, urine or menses) and which are capable of retaining such fluids under moderate pressures. Typically, the particulate, absorbent, polymeric compositions of the present invention will swell and rapidly absorb the fluids with little or no incidence of gel blocking.

The polymeric compositions of the present invention are formed from polymer materials capable of absorbing large quantities of liquids (such polymer materials are commonly referred to as hydrogel, hydrocolloid, super-absorbent materials, or absorbent gelling materials). The polymeric compositions preferably comprise particles of substantially water-insoluble, absorbent, hydrogel-forming, polymer material. The polymer materials useful for the particles of the polymeric compositions may widely vary, but are generally described as polyelectrolytes or are polyelectrolytic in nature.

As used herein, the term “super-absorbent material” refers to a water-swellable, water-insoluble organic or inorganic material capable, under the most favorable conditions, of absorbing at least several times, preferably at least 10 times and most preferably at least 30 times, its weight in an aqueous solution containing 0.9 weight percent of sodium chloride. Organic materials suitable for use as a super-absorbent material of the present invention can include natural materials such as agar, pectin, guar gum, and modified natural materials such as the sodium salt of carboxymethylcellulose, as well as synthetic materials such as synthetic hydrogel polymers. Such hydrogel polymers include, for example, alkali metal salts of polyacrylic acids, partially-neutralized polyacrylamides, ethylene maleic anhydride copolymers, i, and polymers and copolymers of vinyl sulfonic acid, polyacrylates, polyacrylamides, and polyvinyl pyridines. Other suitable polymers include hydrolyzed acrylonitrile grafted starch, acrylic acid grafted starch, and isobutylene maleic anhydride copolymers and mixtures thereof. Examples of polymer materials suitable for use include those which are prepared from polymerizable, unsaturated, acid-containing monomers. Such monomers include the olefinically unsaturated acids and anhydrides which contain at least one carbon to carbon olefinic double bond. More specifically, these monomers can be selected from olefinically unsaturated carboxylic acids and acid anhydrides, olefinically unsaturated sulfonic acids and mixtures thereof. Some non-acid monomers may also be used to prepare the precursor particles herein. Such non-acid monomers can include, for example, the water-soluble or water-dispersible esters of the acid-containing monomers as well as monomers which contain no carboxyl or sulfonic acid groups at all. Optional non-acid monomers can thus include monomers containing the following types of functional groups: esters derived from carboxylic or sulfonic acids, hydroxyl groups, amide-groups, amino groups, nitrile groups and quaternary ammonium salt groups. Olefinically unsaturated carboxylic acid and carboxylic acid anhydride monomers include the acrylic acids typified by acrylic acid itself, methacrylic acid, ethacrylic acid, alpha-chloroacrylic acid, alpha-cyano acrylic acid, beta-methyl acrylic acid (crotonic acid), alpha-phenyl acrylic acid, beta-acryloxy propionic acid, sorbic acid, alpha-chloro sorbic acid, angelic acid, cinnamic acid, p-chloro cinnamic acid, beta-steryl acrylic acid, itaconic acid, citraconic acid, mesaconic acid, glutaconic acid, aconitic acid, maleic acid, fumaric acid, tricarboxyethylene and maleic acid anhydride. Olefinically unsaturated sulfonic acid monomers include aliphatic or aromatic vinyl sulfonic acids such as vinylsulfonic acid, allyl sulfonic acid, vinyltoluene sulfonic acid and styrene sulfonic acid; acrylic and methacrylic sulfonic acid such as sulfoethyl acrylate, sulfoethyl methacrylate, sulfopropyl acrylate, sulfopropyl methacrylate, 2-hydroxy-3-acryloxy propyl sulfonic acid, 2-hydroxy-3-methacryloxy propyl sulfonic acid and 2-acrylamido-2-methyl propane sulfonic acid. Other polymer materials for use in the present invention possess a carboxyl group. These polymers include hydrolyzed starch-acrylonitrile graft copolymer, partially neutralized starch-acrylonitrile graft copolymer, starch-acrylic acid graft copolymer, partially neutralized starch-acrylic acid graft copolymer, saponified vinyl acetate-acrylic ester copolymers, hydrolyzed acrylonitrile or acrylamide copolymers, lightly crosslinked products of any of the foregoing copolymers, partially neutralized polyacrylic acid, and slightly network crosslinked products of partially neutralized polyacrylic acid. These polymers may be used either independently or in the form of a polymeric mixture derived from two or more monomers, compounds, or the like.

While super-absorbents are theoretically provided in various sheet or particulate alternatives, they are efficaciously usually provided as a granular particulate typically sized between 100 microns and 1000 microns. The term “particulate” is used herein to mean that the elements comprising the polymeric composition are in the form of discrete units denominated “particles.” The particles can comprise granules, i, spheres, flakes, fibers, aggregates or agglomerates. Thus, the particles can have any desired shape such as cubic; rod-like; polyhedral; spherical; rounded; angular; irregular; randomly-sized irregular shapes (for example, pulverulent products of a grinding or pulverizing step or aggregates) or shapes having a large greatest dimension/smallest dimension ratio like needle-like, flake-like, or fibrous shapes. The term particles further include aggregates and fibers. As used herein, the term “aggregate” is used to mean a single “particle” formed from two or more previously independent particles (that is, “precursor particles”) joined together. Certain elongated or flaked particles (for example, without limitation, fibers or rod-like particles) are not effectively sized by means of sieving and are sized, when size control is needed, by measurement of the length and width of control samples using optical microscopy.

Although the particles may have sizes varying over a wide range, specific particle size distributions and sizes are preferred. For purposes of the present invention, particle size is defined as the dimension of a particle or precursor particle which is determined by sieve size analysis. Thus, for example, a particle that is retained on a standard #30 sieve with 600 micron openings is considered to have a particle size greater than 600 microns, a particle that passes through the #30 sieve with 600 micron openings and is retained on a standard #35 sieve with 500 micron openings is considered to have a particle size between 500 and 600 microns, and a particle that passes through the #35 sieve with 500 micron openings is considered to have a particle size less than 500 microns. The particles will generally range in size from 100 microns to 2000 microns in diameter or cross-section; preferably, the particles will have a particle size from 100 microns to 1000 microns.

While super-absorbent particles are, in some instances, deployed as a particulate bed, a permeable substruction meshwork (also denoted as a web or matrix) is usually also deployed in the absorbent medium to space the super-absorbent particles from each other, provide cushioning, provide open voids to permeate and transfer liquid through the medium, and provide strength to the composite as a whole. The most commonly used permeable substruction meshwork is cellulose fluff. Cellulose fluff is made of cellulose fibers obtained from wood-pulping processes and is commonly used in absorption applications where strands of the fibers are loosely intertwined to provide a meshwork or web having a greater volumetric percentage of open void than of intertwined stranding (stranding being defined as a plurality of strands loosely woven or otherwise formed into a mesh or webbing with a strand being defined as a flexible and elongated string-form unit and/or internodal flexible and elongated string-form web segment). Synthetic polymers can also be formed into fibers or filaments (fibrous polymers) for meshwork construction. Other naturally-occurring fibrous materials (for example, without limitation, cotton and/or wool) provide further alternative stranding types. In another alternative, a permeable sponge having elongated polymer filament stranding of strand elements whose ends are conjoined at connection nodes provides a meshwork. In yet another alternative, a permeable sponge of mechanically woven elongated polymer filament provides a meshwork. In a further alternative, a foamed sponge provides a meshwork.

It is to be noted that meshworks frequently have an absorption capability without the benefit of super-absorbent particles being intermixed within them. In this regard, natural and manufactured sponges (open-celled, elastic porous masses of synthetic or natural fibers capable of absorbing water or aqueous fluids) are well-known for non-tightly bonding liquids for an interim period and are widely used in household cleaning as well as in personal washing and bathing. In this regard, an absorption capacity value (the mass of liquid absorbed per unit mass of stranding component measured by first saturating a known quantity of stranding component with liquid, blotting the saturated stranding component under standard conditions to remove unabsorbed liquid, and measuring the increase in mass of the stranding component) is useful in characterizing the ability of a meshwork to absorb liquid.

When super-absorbent particles are well intermixed throughout the volume of the meshwork to form the absorbent medium of the composite, the particles adhere-to or are physically entrapped-by the stranding of the meshwork and are effectively kept in a dispersed orientation throughout the meshwork by their adherence-to or entrapment-by the stranding; in this regard, the meshwork provides both (a) structural support for the composite as a whole and (b) a substruction within the intermixed absorbent medium for maintaining the dispersed super-absorbent particles throughout the meshwork.

Fibers suitable for use in the permeable substruction meshwork (also denoted as a web or matrix) of the present invention include cellulosic fibers such as wood pulp fluff, cotton, and cotton linters, as well as synthetic polymeric fibers including modified cellulose fibers, rayon, polypropylene, and polyester fibers such as polyethylene terephthalate (DACRON™), hydrophilic nylon (HYDROFIL™), cellulose acetate, acrylics, polyvinyl acetate, polyamides (such as nylon), multicomponent fibers, and mixtures thereof. Hydrophilic fiber materials are preferred. Examples of suitable hydrophilic fiber materials in addition to some already mentioned are hydrophilized hydrophobic fibers, such as surfactant-treated or silica-treated thermoplastic fibers derived, for example, from polyolefins such as polyethylene or polypropylene, polyacrylics, polyamides, polystyrenes, and polyurethanes. Other cellulosic fiber materials which may be useful in certain absorbent members herein are chemically stiffened cellulosic fibers. Chemically stiffened cellulosic fibers are also efficacious as stiffened, twisted, curled cellulosic fibers which can be produced by internally crosslinking cellulose fibers with a crosslinking agent.

Further details in super-absorbent and meshwork materials are presented in WO 99-17694, U.S. Pat. No. 5,330,822, and U.S. Pat. No. 5,843,059.

Turning now to FIG. 1, an architectural model of an absorbent composite 100 is presented. Composite 100 has an upper tissue cover 102 and a lower tissue cover 104 with tissue covers 102, 104 each providing a thin, porous and wettable wrap enclosing absorbent medium 106 and generally providing additional strength to medium 106 when wet. Medium 106 is further made of intertwined stranding 108 defining a substruction meshwork (web) for holding super-absorbent polymer (SAP) particles 110 in a dispersed manner throughout medium 106. Stranding 108 is intertwined to provide sufficient void space so that the meshwork is a permeable substruction meshwork providing rapid fluid communication of liquid transferred though tissue cover 102 to super-absorbent polymer particles 110. The collection of all super-absorbent polymer particles 110 define a polymer component portion within medium 106 and the collection of all intertwined stranding 108 defines a stranding component portion within medium 106. In the depicted alternative, the permeable substruction meshwork is made of stranding arranged to provide permeable transfer; accordingly, the depicted permeable substruction stranding is a more specific alternative of permeable substruction meshwork. In an alternative embodiment, the permeable substruction meshwork is a permeable sponge of elongated polymer filaments whose ends are conjoined at connection nodes. In a further alternative, a foamed sponge provides the permeable substruction meshwork. In yet another alternative, a permeable sponge of mechanically woven elongated polymer filament provides the permeable substruction meshwork. The preferred specific embodiment of permeable substruction stranding is cellulose fluff.

Respective to the medium 106 as a distinctive domain, a modeled context is now presented for providing the basis for the particular features of the present invention as further described herein.

Absorbent medium 106 is comprised of the meshwork 108, the super-absorbent polymer particles 10, and any pore spaces between them. The pores are filled with air in the dry state and are filled generally with a mixture of air and liquid that is unabsorbed by either the fibers or the super-absorbent polymer particles in the wet state. A mass balance of liquid in the absorbent medium indicates that the mass of liquid that is absorbed by any given dry mass of absorbent medium is taken as the sum of the masses of liquid absorbed by the individual fibers, the super-absorbent polymer particles and the pore spaces according to the following equation
m_{liq} =mₚₒᵣₑₛ+C_{fiber}m_{fiber}+γmₛₐₚ (1)
where m_{liq} is the cumulative mass of all the liquid in the absorbent medium, mₚₒᵣₑₛ is the mass of liquid in pores, C_{fiber} is the specific absorptivity of the fiber substance (denoted herein as the absorption capacity value of the fiber), m_{fiber} is the cumulative mass of all fibers used in the medium, γ is the specific absorptivity of the super-absorbent polymer and mₛₐₚ is the cumulative mass of all super-absorbent polymer particles in the medium. In the present invention, the absorbent medium is considered perfectly dry when the mass of liquid in the pores is zero. Equation (1) can be therefore simplified to
m_{liq}=C_{fiber}m_{fiber}+γmₛₐₚ. (2)

As noted in the background section, the absorption capacity of super-absorbent polymers conventionally is measured by the centrifuged retention capacity (CRC) test. The CRC value conventionally is identified with the value of the specific absorptivity γ of the super-absorbent polymer. It is a surprising discovery that this is an inadequate measure of the specific absorptivity of the super-absorbent polymer during realistic usage. When the CRC is used as the measure of specific absorptivity during realistic usage, the liquid applied cannot completely be absorbed by the fiber substance and the super-absorbent polymer, and the absorbent medium is wet. The specific absorptivity of the super-absorbent polymer is written as the mathematical product of the CRC and a dryness quality value Φ according to the following equation
γ=ΦCRC (3)
where the numerical value of Φ conventionally is taken as 1. Equation (2) is rewritten to include (3) as follows
m_{liq}=C_{fiber}m_{fiber}+ΦCRCmₛₐₚ (4)

In addition, the total dry mass of the absorbent medium is the sum of the masses of the fiber meshwork and the super-absorbent polymer particles according to the following equation
m_{T}=m_{fiber}+mₛₐₚ (5)
which can be rearranged to the following mathematical identity

$$1 = \frac{{m}_{fiber}}{{m}_{T}} + \frac{{m}_{sap}}{{m}_{T}} \qquad ( 6 )$$

Defining fₛ as the ratio of the mass of dry super-absorbent polymer cumulation to the mass of the dry absorbent medium (that is, fₛ is the mass fraction of super-absorbent polymer particles in the dry absorbent medium), the previous equation can be rearranged to provide an expression for the mass fraction of dry fibers in terms of fₛ as follows

$$\frac{{m}_{fiber}}{{m}_{T}} = 1 - {f}_{s} \qquad ( 7 )$$

These expressions for the mass fractions of the dry components can be rearranged to provide expressions for the mass of each component in terms of the total dry mass of the absorbent medium and the mass fraction of super-absorbent polymer particles as follows
m_{fiber}=m_{T}(1−fₛ) (8)
mₛₐₚ=m_{T}fₛ (9)

Substituting (8) and (9) into (4) yields the following equation
m_{liq}=C_{fiber}m_{T}(1−fₛ)+ΦCRCm_{T}fₛ (10)
which can be rearranged to yield an expression for the total mass of absorbent medium necessary to absorb the mass of liquid

$${m}_{T} = \frac{{m}_{liq}}{{C}_{fiber} ( 1 - {f}_{s} ) + ΦCRC {f}_{s}} \qquad ( 11 )$$

The porosity of an absorbent medium can be described in the following context. Absorbent medium 106 is comprised of the intertwined fibers or meshwork 108, the super-absorbent polymer particles 110, and any pore spaces between them. These pores are filled with air in the dry state and are filled generally with a mixture of air and liquid that is unabsorbed by either the fibers or the super-absorbent polymer particles in the wet state. The total volume of the absorbent medium is given by the sum of the volumes of the individual components as follows
V_{T}=Vₚₒᵣₑₛ+V_{fibers}+Vₛₐₚ (12)
which can be mathematically rearranged to provide the following equation

$$1 = \frac{{V}_{pores}}{{V}_{T}} + \frac{{V}_{fibers}}{{V}_{T}} + \frac{{V}_{sap}}{{V}_{T}} \qquad ( 13 )$$

wherein the individual volume ratios represent the volume fraction of pores, the volume fraction of fibers and the volume fraction of super-absorbent polymer particles, respectively. The porosity of the absorbent medium is defined as the volume fraction of pores, hence the equation can be rewritten as follows

$$ϕ = 1 - \frac{{V}_{fibers}}{{V}_{T}} - \frac{{V}_{sap}}{{V}_{T}} = 1 - \frac{( {V}_{fibers} + {V}_{sap} )}{{V}_{T}} \qquad ( 14 )$$

The volume of fibers present in the absorbent medium is given by their total mass divided by their density. In the event that the particular fibrous substance chosen absorbs some liquid, that absorption can be characterized by a fiber absorption capacity C_{fiber}. Then the volume of fibers in the wet state is given by the sum of the dry volume of fiber and the volume of liquid absorbed according to the following equation

$${V}_{fibers} = \frac{{m}_{fiber}}{{ρ}_{fiber}} + \frac{{m}_{fiber} {C}_{fiber}}{{ρ}_{liq}} \qquad ( 15 )$$

The volume of super-absorbent polymer particles present in the absorbent medium is given by their total mass divided by their density. In the event that the particles are swollen with liquid, that absorption can be characterized by a swelling ratio Q, which is the ratio of the mass of liquid absorbed by the particles divided by their dry mass. Then the volume of the super-absorbent polymer particles in the wet state is given by the sum of the dry volume of super-absorbent polymer particles and the volume of liquid absorbed by the super-absorbent polymer according to the following equation

$${V}_{sap} = \frac{{m}_{sap}}{{ρ}_{sap}} + \frac{{m}_{sap} Q}{{ρ}_{liq}} \qquad ( 16 )$$

The expression of the mass of dry fibers from (8) is substituted into (15), and the expression for the mass of dry super-absorbent polymer cumulation from (9) is substituted into (16), then the modified versions of (15) and (16) are substituted into (14) to yield the following expression for the porosity of the uncompressed composite when wet (φ₀)

$$\begin{matrix}{ϕ}_{0} = 1 - \frac{( {V}_{fibers} + {V}_{sap} )}{{V}_{T}} \\ = 1 - \frac{{m}_{T}}{{V}_{T}} [ ( 1 - {f}_{s} ) ( \frac{1}{{ρ}_{fiber}} + \frac{{C}_{f}}{{ρ}_{liq}} ) + {f}_{s} ( \frac{1}{{ρ}_{sap}} + \frac{Q}{{ρ}_{liq}} ) ]\end{matrix} \qquad ( 17 )$$

In equation (17), the quantity in the square brackets describes how the mass of the components increases, or scales up, when the fibers and super-absorbent polymer particles absorb liquid. In addition to the change in mass of the absorbent medium during absorption and swelling, the volume of the composite V_{T} also changes. But the volume of the composite may not change to the same degree as the mass of the components, thereby leading to a possible change in porosity during absorption and swelling. The inventors have made the surprising discovery that the volume change of the composite scales up by a factor having the same terms as in the square brackets of equation (17) but raised to a power much smaller than that for the mass change. In mathematical terms, the volume of the wet composite is scaled up from the volume of the dry composite according to the following equation

$${V}_{T} = {{V}_{Tdry} [ ( 1 - {f}_{s} ) ( \frac{1}{{ρ}_{fiber}} + \frac{{C}_{f}}{{ρ}_{liq}} ) + {f}_{s} ( \frac{1}{{ρ}_{sap}} + \frac{Q}{{ρ}_{liq}} ) ]}^{q} \qquad ( 18 )$$

where the value of the exponent q depends on the specific type of fibrous meshwork employed. Substituting this expression for V_{T} into equation (17) yields the following equation for the porosity

$${ϕ}_{0} = 1 - {\frac{{m}_{T}}{{V}_{Tdry}} [ ( 1 - {f}_{s} ) ( \frac{1}{{ρ}_{fiber}} + \frac{{C}_{f}}{{ρ}_{liq}} ) + {f}_{s} ( \frac{1}{{ρ}_{sap}} + \frac{Q}{{ρ}_{liq}} ) ]}^{m} \qquad ( 19 )$$

where the exponent m=1−q. As should be apparent, the quantity m_{T}/V_{Tdry} is the bulk density of the dry absorbent composite. For most situations of interest, the first term of the sum inside the square brackets (fiber term) is small compared to the second term (super-absorbent polymer term) and can be neglected, which yields the following simplified equation for the porosity of composites containing super-absorbent polymer

$${ϕ}_{0} = 1 - {\frac{{m}_{T}}{{V}_{Tdry}} [ {f}_{s} ( \frac{1}{{ρ}_{sap}} + \frac{Q}{{ρ}_{liq}} ) ]}^{m} \qquad ( 20 )$$

The forgoing discussion of porosity of absorbent composites applies in the absence of externally applied compression, such as is applied by an infant sitting on a diaper containing such composite. In modeling respective to the dependence of porosity of super-absorbent composites to pressure from such externally applied compression, similarities of such composites to foams is of value. In this regard, a work by S. Swyngedau, et al., entitled “Models for the Compressibility of Layered Polymeric Sponges”, Polymer Engineering and Science, Volume 31, number 2, pages 140-144 (1991). Drawing upon the relationship as modeled on page 141 in that work (Model A), the inventors postulated a modification according to Equation 21 and, via further empirical effort, have made the surprising discovery that the compressibility of such swollen composites is understood, and therefore designed, in terms of a simple scaling function of the applied pressure P and the elastic modulus of the swollen gel G, wherein the scaling exponent of the function depends on the mass fraction of super-absorbent polymer particles in the absorbent composite. Mathematically this is stated by the following equation describing the ratio R_{φ} of the porosity of a composite under a compression P (denoted herein as φ) to the uncompressed porosity φ₀

$${R}_{ϕ} = \frac{ϕ}{{ϕ}_{0}} = \frac{1}{{( 1 + P / G )}^{n}} \qquad ( 21 )$$

where the exponent n determines the sensitivity of the composite to compression. The optimum porosity behavior of the pad is defined in terms of the retention of porosity in the pad during any compression. The inventors have made the surprising discovery that the value of the exponent n depends on the mass fraction of super-absorbent polymer in the composite, according to the essentially smooth function depicted in FIG. 3, and that, accordingly, the trend of the measurements is efficaciously described by
n=fₛ^{1.83}+0.07 (22)

Incorporating this feature into equation (20) yields the following expression for the porosity of a composite

$$ϕ = \frac{1 - {\frac{{m}_{T}}{{V}_{Tdry}} [ {f}_{s} ( 1 / {ρ}_{sap} + Q / {ρ}_{liq} ) ]}^{m}}{{( 1 + P / G )}^{n}} \qquad ( 23 )$$

Rearranging the equation for the porosity ratio R_{φ} yields an equation for the shear (elastic) modulus of the polymer needed to provide the desired porosity under compression P (units of P and G identical). This is called the critical shear modulus at compression P.

$$G = \frac{P}{[ {( \frac{{ϕ}_{P0}}{{ϕ}_{P}} )}^{1 / n} - 1 ]} = \frac{P}{[ {( 1 / {R}_{ϕ} )}^{1 / n} - 1 ]} \qquad ( 24 )$$

A reference pressure is arbitrarily chosen, P=20684.3 dynes/cm² (0.3 psi) to provide a numerical basis for final calculation. It is apparent to those skilled in the art that the porosity ratio will take on other values when the pressure is other than 0.3 psi. The value of the porosity ratio at other pressures is determined from the following relationship, which has been derived from equation (24).

$$\frac{1}{{R}_{ϕ2}} = {\{ 1 + \frac{{P}_{2}}{{P}_{ref}} [ {( 1 / {R}_{ϕ ref} )}^{1 / n} - 1 ] \}}^{n} \qquad ( 25 )$$

wherein R_{φ2} is the new porosity ratio when pressure is changed to P₂ from P_{φref}. The exponent n is given by equation (22).

The relationship between the necessary, or critical shear (elastic) modulus from equation (24), and the corresponding value of the swelling ratio (when saturated) has been found by experimentation (FIG. 4) to be

$$CRC = \frac{8600}{{G}^{0.54}} \qquad ( 26 )$$

where the CRC units are g/g and the G units are dynes/cm². The CRC actually used in the composite must be no greater than this value (at the chosen fₛ value) for the porosity criterion to be satisfied.

Combining equations (22), (24) and (26) yields an expression for the maximum value of the swelling ratio CRC that is useful in providing desired porosity in a composite with composition given by fₛ

$$CRC = {40.58 [ {( \frac{1}{{R}_{ϕ}} )}^{\frac{1}{{f}_{s}^{1.83} + 0.07}} - 1 ]}^{0.54} \qquad ( 27 )$$

An optimally dry and optimally porous absorbent medium can be described by substituting the expression for CRC from equation (27) into the expression for the total dry mass of composite, equation (11), to yield the following expression

$${m}_{T} = {m}_{liq} / \{ ( 1 - {f}_{s} ) {C}_{fiber} + 40.58 {f}_{s} {Φ [ {( \frac{1}{{R}_{ϕ}} )}^{\frac{1}{{f}_{s}^{1.83} + 0.07}} - 1 ]}^{0.54} \} \qquad ( 28 )$$

Turning now to empirically defined considerations in the present invention, FIGS. 2, 3, and 4 present results from laboratory work which relate-to and define key factors in the dryness quality value, the porous quality value, and specific values in the equations

$$CRC = {F [ {( \frac{1}{{R}_{ϕ}} )}^{\frac{1}{{f}_{s}^{1.83} + 0.07}} - 1 ]}^{0.54}$$

$$and$$

$${m}_{total} = \frac{{m}_{liq}}{\{ ( 1 - {f}_{s} ) {C}_{stranding} + {Ff}_{s} {Φ [ {( \frac{1}{{R}_{ϕ}} )}^{\frac{1}{{f}_{s}^{1.83} + 0.07}} - 1 ]}^{0.54} \}} .$$

FIG. 2 presents graphical information respective to the dryness quality value. The dryness quality value concept is derived from the measurements of SAP swelling in a quantity of liquid equal to the product of the CRC of the polymer times the mass of SAP used in the test times a liquid fraction value. First the swelling extent of the SAP is measured in an excess bath of liquid according the centrifuged teabag test, to determine the CRC value of the polymer. Then a new sample of the same polymer is used to measure the swelling extent when only a limited amount of saline is added. The limited amount of saline is varied according to the above mentioned liquid fraction value to measure the response of the polymer to varying quantities of liquid. For polymers having different CRC values, the results of swelling in limited quantities of liquid can be normalized by reporting the ratio of swelling extent in limited liquid quantity to swelling extent in excess liquid quantity (CRC value). The tested polymers tended to fall along a single curve as shown in FIG. 2. The normalized ratio defines the dryness quality values where the ratio is smaller than the expected absorption ratio.

In a surprising discovery, under the real conditions of use of absorbent products such as diapers, super-absorbent polymers do not absorb the amount of liquid as determined by the traditional CRC method. All tested super-absorbent polymers absorb much less liquid than that predicted by the traditional CRC method. The amount absorbed under realistic conditions (a limited amount, rather than an “infinite” supply or large excess of liquid) falls into the range of 45-85 percent of the CRC value. Note that the reciprocal of 0.45 (45 percent) is a value of 2.22 and that the reciprocal of 0.85 (85 percent) is a value of 1.18. Therefore, absorbent designs of current practice rely on the CRC value whereas those of the invention rely on a specified fraction between 45 percent and 85 percent of the CRC value. This important difference allows composites according to the present invention to tightly bind all the liquid delivered to the composite, and therefore exhibit superior dryness compared to current absorbent composites based upon an unmodified CRC value. Therefore, the absorption design-instance parameter is multiplied by between a 1.18 and a 2.22 to define the appropriate mass of super-absorbent polymer cumulation depending upon the full degree of tactile dryness desired. As should be apparent from a consideration of FIG. 2, if a mass of super-absorbent polymer cumulation in excess of a 2.22 multiple of the absorption design-instance parameter is deployed for a given target amount of liquid to be absorbed, then the dryness quality will not be effectively improved beyond that achievable with the 2.22 multiple and absorbent medium 106 will, accordingly, not provide a sufficiently minimal amount (that is, an economically efficient amount given the tactile dryness desired) of super-absorbent polymer for effectively minimizing free aqueous liquid within the super-absorbent polymer cumulation after the targeted weight of aqueous liquid has been absorbed to achieve the result that the super-absorbent polymer cumulation with the targeted weight of absorbed aqueous liquid provides sustained tactile dryness. In this regard, FIG. 2 shows that a mass of super-absorbent polymer cumulation in excess of a 2.22 multiple of the absorption design-instance parameter deployed for a given target amount of liquid to be absorbed is indeed “overkill” for achieving tactile dryness. As should also be apparent from a consideration of FIG. 2, if a mass of super-absorbent polymer cumulation less than a 1.18 multiple of the absorption design-instance parameter is deployed for a given target amount of liquid to be absorbed, then free liquid will be present in medium 106 and a minimal dryness quality will not realistically be achieved and the absorbent medium will, accordingly, not provide a sufficiently minimal amount of super-absorbent polymer for effectively minimizing free aqueous liquid within the super-absorbent polymer cumulation after the targeted weight of aqueous liquid has been absorbed to achieve the result that the super-absorbent polymer cumulation with the targeted weight of absorbed aqueous liquid provides sustained tactile dryness. In this regard, FIG. 2 shows that a mass of super-absorbent polymer cumulation less than a 1.18 multiple of the absorption design-instance parameter deployed for a given target amount of liquid to be absorbed is insufficient for achieving desired tactile dryness. However, when the mass of super-absorbent polymer cumulation is derived from the targeted weight of aqueous liquid by multiplying the absorption design-instance parameter by between 1.18 and 2.22 (depending on the degree of tactile dryness desired respective to the absorbent medium after absorption of the target liquid amount) of the design-instance parameter, a sufficiently minimal amount (that is, the economic amount) of super-absorbent polymer is provided for effectively minimizing free aqueous liquid (that is, effectively achieving the degree of tactile dryness desired respective to the absorbent medium after absorption of the target liquid amount) in the absorbent medium after the targeted weight of aqueous liquid has been absorbed to achieve the result that the super-absorbent polymer cumulation with the targeted weight of absorbed aqueous liquid provides sustained tactile dryness.

The following examples further define the basis for FIGS. 2, 3, and 4.

### EXAMPLE 1

A sample set of 20 super-absorbent polymers was chosen to provide materials from various experimental chemistries and crosslink densities as well as from several commercial sources, and the CRC determined for each of the 20 samples according to the following procedure.

By way of introduction to the procedure, a small sample of polymer was sealed inside a tea bag, immersed in salt water for thirty minutes, and centrifuged to removed unabsorbed liquid. The ratio of the mass of solution absorbed to the initial mass of the polymer was the centrifuge retention capacity (CRC). Most samples in the specified size range essentially reached their maximum swelling capacity in the 30-minute absorption time of the test. For unusually slow-absorbing samples, the absorption time was extended past 30 minutes as necessary to assure that the particular sample essentially reached its maximum swelling capacity.

In detail, the superabsorbent polymer sample was sieved (U.S. Standard Sieves or equivalent) to obtain the fraction passing through a 30 mesh (600 micron) sieve and retained on a 50 mesh (300 micron) sieve, in order to minimize differences in absorption rate caused by differences in the particle size distributions of samples. Tea bag paper (heat sealable, 6.35 cm wide: K-C Grade 542, or equivalent, available from Kimberly-Clark Co., 2100 Winchester Rd., Neenah, Wis. 54956) was cut into a 12.7 cm long strip and folded in half to form a 6.35×6.35 cm rectangle with the sealable surface of the paper inward. Two of the three open sides were sealed (ca. 0.635 cm wide seams) with a hot clothes iron or equivalent heat sealer. The empty tea bag was labeled and weighed (analytical balance: capable of measuring 0.001 g, Mettler Model PM460, or equivalent, available from Mettler Instrument Corp., Princeton-Hightstown Road, Hightstown, N.J. 08520). The mass was recorded as W1. The sample of polymer (0.200 g±0.005 g) was added into the tea bag and the mass of the sample plus the tea bag was recorded as W2. The bag was sealed with the heat sealer and held horizontally to distribute the polymer evenly throughout the bag. Two empty bags were prepared for each sample or batch of samples to use as blanks. A stainless steel utility tray (39×24.8×6.35 cm), available from Fisher Scientific Company, or equivalent container) was filled ¾ full with 0.9 percent mass percent NaCl solution. The sample bags and the blanks were placed on top of a section of polymer-coated fiberglass screen (ca. 0.635 cm openings, 35.6×20.3 cm, available from Taconic Plastics Inc., Petersburg, N.Y.) and another section of screen was placed on the bags. The assembly slowly was lowered into the tray filled with NaCl solution and the timer was started (Lab timer: 30 minute capability, readable to 1 second, available from Fisher Scientific Company). After thirty minutes, the assembly was removed from the NaCl solution. Using tongs, the bags were placed into the centrifuge basket in opposing pairs (the two blanks must be opposite each other) to balance the centrifuge (Centrifuge: capable of a speed of 1500 rpm, Dynac II model, or equivalent, available from Fisher Scientific Company). After the lid was closed, the centrifuge was started and operated for three minutes after a speed of 1500 rpm had been reached. After three minutes, the centrifuge brake was applied to stop the basket. The blanks were removed from the centrifuge with tongs and weighed. The average mass was recorded as B1. The sample bag was removed from the centrifuge and weighed, and the mass was recorded as W3.

The centrifuge retention capacity (CRC) was calculated as follows:

$$CRC ( g \text{/} g ) = \frac{( W3 - B1 ) - ( W2 - W1 )}{W2 - W1}$$

where:
CRC=Centrifuge retention capacity,
W1=Dry mass of empty sample tea bag,
W2=Dry tea bag and sample mass,
W3=Wet tea bag and sample mass, and
B1=Average wet blank tea bag mass.

Data obtained by this procedure indicated a relative standard deviation of 0.65 percent at an average centrifuge retention capacity of 28.6 g/g. The values might be expected to vary from the average by not more than +1.43 percent relative at the 95 percent confidence level.

The fundamental properties of the samples are given in Table 1 below. A graph of the CRC values versus the shear modulus values, which properties were interrelated by virtue of the crosslink density of the samples, is shown in FIG. 4.

TABLE 1
Sample Set of Superabsorbent Polymers Used in This Work

| | CRC | Shear Modulus |
|---|---|---|
| Sample ID | (g/g) | (dynes/cm²) |
| Drytech 2035 (Dow Chemical) | 29.2 | 42000 |
| Experimental polyacrylate AFA173-38 | 38.4 | 22000 |
| Experimental polyacrylate AFA176-107HT | 37.3 | 39300 |
| Drytech 535 (Dow Chemical) | 29.6 | 40100 |
| Favor 880 (Stockhausen GmbH) | 29.4 | 31800 |
| Favor SXM 7500 (Stockhausen GmbH) | 37.7 | 30100 |
| Aqualic CAW4 (Nippon Shokubai K.K.) | 35.1 | 26200 |
| Experimental polyacrylate ST | 42.8 | 13700 |
| ASAP 2300 (Chemdal Inc.) | 28.0 | 41300 |
| Sanwet IM 4510 (Hoechst Celanese Corp.) | 30.7 | 43800 |
| Experimental polyacrylate AFA173-133 | 22.0 | 50571 |
| Experimental polyacrylate AFA202-103 | 14.7 | 90300 |
| Experimental polyacrylate AFA203-32-4 | 27.7 | 47600 |
| Experimental polyacrylate AFA173-113 | 24.7 | 40100 |
| Experimental polyacrylate AFA173-105 | 18.1 | 74600 |
| Experimental polyacrylate XZ-91060.02 | 27.4 | 38200 |
| Drytech 2024 (Dow Chemical) | 27.4 | 60800 |
| Experimental polyacrylate AFA210-15 | 38.8 | 28300 |
| Experimental polyacrylate AFA207-52D | 32.6 | 33500 |
| Experimental polyacrylate AFA207-52C | 33.0 | 25200 |

The shear modulus of super-absorbent polymer (SAP Modulus) was measured on packed beds of swollen particles. The swelling extent Q_{comp} of each super-absorbent polymer was determined from swelling experiments in the presence of cellulose fiber as outlined in the section “Saturation and Blotting Technique.” A fresh sample of the super-absorbent polymer was then prepared by adding the amount of 0.9 percent NaCl solution required by Q_{comp} to a known quantity of the polymer (30-50 mesh particles), letting the polymer absorb the saline solution for 60 minutes, and then measuring the shear modulus on a packed bed of gel according to our previous description of the technique. The resulting values are tabulated in Table 1 above.

### EXAMPLE 2

To determine the swelling extent of super-absorbent polymer when only a limited amount of liquid is available, the Limited Tea Bag Swelling test was used. 30-50 mesh cuts of the granular super-absorbent polymer samples were isolated and the standard centrifuge retention capacity value for each sample was then measured using a pair of tea bags in a very large excess of 0.9 percent NaCl solution. Then, for each sample, a pair of tea bags (essentially identical to those used for the comparable CRC measurement) containing 0.2 g of super-absorbent polymer was set up. Each tea bag was placed into a 8.89 cm diameter Petri dish and then saline solution was added to the tea bag. The amount of saline equaled the mass of polymer in the bag times its CRC value times the fractional swelling desired, plus 0.35 grams extra saline needed to wet the tea bag. The dish was covered and the tea bag was left for 60 minutes. Then the matched pair of tea bags was centrifuged in exactly the same way as for standard CRC. The bags were weighed and the swelling capacity of the super-absorbent polymer was calculated in the usual fashion. The swelling extent of each polymer was measured with saline volumes equal to liquid fraction values of 0.4, 0.6, 0.8, 1.0 and 2.0 times the CRC value. The normalized swelling value was calculated by dividing the swelling extent measured in limited liquid volume by the swelling extent measured in a large excess of liquid (CRC value).

In FIG. 2, the normalized swelling value is plotted versus the liquid fraction value. It is clear that all the polymer samples tested absorbed limited volumes of liquid in a similar way.

At each liquid fraction value, the polymers swell to a value that was less than the CRC value; the normalized swelling values are all smaller than unity. All the SAPs, irrespective of their crosslinking chemistry or crosslinking extent, behaved in a similar way.

Also drawn on FIG. 2 is a straight line segment representing the conventional “expectation” that the swelling should equal the value computed by multiplying the CRC by the respective liquid fraction value, for liquid fraction values less than or equal to 100 percent. For liquid fraction values greater than 100 percent, the conventional expectation is that the polymer will absorb liquid equal to the CRC value. By comparing the measurements to the straight line “expectation”, the graph shows that SAPs will absorb all the liquid applied to them when insulted at 40 percent or less of their CRC, but the measured absorption is further from the expectation line as more liquid is added. The measured absorption is furthest from the expectation line at insults equal to 100 percent of the CRC. As liquid is added beyond 100 percent, the SAP becomes flooded in a great bath of saline, and the swelling of SAP gradually approaches the CRC value. The measured absorption improves, but at the expense of a large excess of unabsorbed liquid. However, the swelling does not reach the CRC value until the liquid amount added is greater than twice the CRC value times the mass of polymer.

Pad construction: Composite pads of cellulose fluff and super-absorbent polymer were made in a pad former, using 1.00 g of super-absorbent polymer, 1.00 g cellulose fluff and a tissue paper cover on top and bottom of the 7.62 cm diameter pad. The pad was heat compressed in a Dake hydraulic press heated to 100 degrees C., using spacer shims of 0.318 cm thickness. Three separate polymer samples were used to demonstrate the invention. The samples differed principally in their crosslink density, which controls the maximum amount of 0.9 percent sodium chloride solution that the polymers can absorb. The maximum amount of absorption was determined for each sample using the industry-standard centrifuge retention capacity test. Pads made using this CRC value of the SAP as the basis for construction were control examples. Pads made using values of 60 percent or 80 percent of the CRC values were examples of this invention. Other inventive pad structures can be made with the use of equation (11).

Pad wetting: Each pad was placed into a plastic Petri dish and was wetted with the calculated amount of 0.9 percent sodium chloride solution, which was determined by the mathematical product of the mass of super-absorbent polymer in the pad times its CRC value. The Petri dish was covered and the pad was left to stand for 60 minutes at room temperature.

Determination of unabsorbed liquid by blotting: After the prescribed standing time, the pads were blotted according to the following procedure to remove and measure the unabsorbed liquid. Each approx. 7.62 cm diameter swollen pad was sandwiched between 8 stacked disks of 7.62 cm diameter blotter card (total dry mass of blotter cards is 12.5 g). Four of the disks were placed against the bottom surface of the swollen pad, and four disks were placed against the top surface of the swollen pad. Then a 5 kg weight was placed on top of the sandwich and left in place for 5 minutes. The liquid in the swollen pad that is unabsorbed by the fluff or the super-absorbent polymer was taken up into the porous structure of the blotter cards. The cards were taken away from the top and bottom surfaces of the pad, and the pad mass was measured in the wet and blotted condition (“blotted” referencing the essentially complete mass transfer loss by the pad of that essentially free liquid previously within the pad which could be transferred to the blotter cards in the above procedure). The quantity of unabsorbed liquid was calculated by the difference of the mass of liquid initially added to the pad minus the net absorption by the pad after blotting. The dryness of the pads could be compared by using either of two methods. A partition coefficient could be defined as the ratio of the mass of unabsorbed liquid to the mass of the absorbed liquid. Thus, a small value of the partition coefficient was desirable for drier absorbent structures. Alternatively, a free saturation value for the pad could be defined as the ratio of the mass of unabsorbed liquid to the mass of the swollen and blotted pad. Table 2 below shows the results. The inventive compositions have improved dryness as indicated by smaller values of the free saturation and of the liquid partition coefficient, compared to the pads made by the conventional methodology.

TABLE 2

| Polymer | | | design | un- | | | Total | | |
|---|---|---|---|---|---|---|---|---|---|
| CRC, | pad | | capacity, | absorbed | absorbed | Part. | wet | “Free | |
| g/g | no. | φ | g | liquid, g | liquid, g | Coeff. | disk, g | Saturation” | |
| 22 | 1 | 0.6 | 13.22 | 0.54 | 12.61 | 0.04 | 14.88 | 0.036 | invention |
| 22 | 2 | 0.8 | 17.6 | 0.84 | 16.7 | 0.05 | 18.97 | 0.044 | invention |
| 22 | 3 | 1 | 22.03 | 4.79 | 17.18 | 0.28 | 19.31 | 0.248 | control |
| 22 | 4 | 1.3 | 28.03 | 9.07 | 18.89 | 0.48 | 21.11 | 0.43 | control |
| 29 | 1 | 0.6 | 15.77 | 0.41 | 15.27 | 0.03 | 17.4 | 0.024 | invention |
| 29 | 2 | 0.8 | 21.55 | 1.73 | 19.74 | 0.09 | 21.91 | 0.079 | invention |
| 29 | 3 | 1 | 29.4 | 7.75 | 21.58 | 0.36 | 23.75 | 0.326 | control |
| 29 | 4 | 1.3 | 38.22 | 13.72 | 24.42 | 0.56 | 26.59 | 0.516 | control |
| 38 | 1 | 0.6 | 22.02 | 0.85 | 21.09 | 0.04 | 23.23 | 0.037 | invention |
| 38 | 2 | 0.8 | 29.47 | 4.19 | 25.17 | 0.17 | 27.3 | 0.153 | invention |
| 38 | 3 | 1 | 38.31 | 12.08 | 26.16 | 0.46 | 28.31 | 0.427 | control |
| 38 | 4 | 1.3 | 48.33 | 17.54 | 30.65 | 0.57 | 32.83 | 0.534 | control |

Several comparisons of pads made using the “control” method of design capacity versus the inventive method of design capacity are presented in Table 2. A pad made with a control design capacity of 22 g liquid is pad 22-3, with a free saturation of 0.248, which may be compared to a pad with an inventive design capacity of 22 g liquid, pad 38-1, with a free saturation of 0.037. A pad made with a control design capacity of 29.4 g liquid is pad 29-3, with a free saturation of 0.326, which may be compared to inventive pad 38-2, with a free saturation of 0.153.

### EXAMPLE 3

As previously noted, while the absorbent medium in one embodiment was a bed of super-absorbent particles without the benefit of a substruction meshwork, in an alternative embodiment, the absorbent medium usually included a permeable substruction meshwork through which the super-absorbent material was dispersed. Porosity was frequently a consideration in the permeability of meshworks having dispersed super-absorbent, and, in a second surprising find, the inventors have made a second set of related surprising discoveries that, when the composite absorbed liquid, the volume change of the composite scaled up by a factor much smaller than that of the mass change and that, under compressive pressure, the compressibility of the swollen composite was efficaciously modeled terms of a straightforward scaling function of (a) the applied pressure and (b) the elastic modulus of the swollen gel, with the scaling exponent of the function depending on the mass fraction of super-absorbent polymer particles in the absorbent composite according to an essentially smooth function. The inventors had further discovered that, for an optimally porous composite, there existed a critical value of the CRC above which the composite exhibited poor porosity in the swollen state. The importance of that is understood by reprising the Background statement of contrasting current practice in the hygiene products industry where (a) the porosity of liquid permeability of composites that contain super-absorbent polymer were correlated to the liquid permeability of a granular bed of the swollen super-absorbent polymer and (b) that the design of the composite was based on this correlation. Details in this second surprising find are further appreciated from a consideration of FIG. 3 and of the following work which further defines the basis for FIG. 3.

In addition to the specification of composite design set forth above in section 5, a porosity criterion for the swollen pad was imposed to make preferred composite structures. The inventors studied the manner in which the porosity of the swollen pads depended on the nature and amount of the swollen gel present in the composite structure and found that the porosity followed a scaling law in the fraction of gel present in the structure, according to equation (25). This equation stated that the porosity of the swollen pad decreased from its initial dry value depending on the amount of gel present in the swollen pad. As the amount of gel increased, the porosity decreased. Low values of porosity were undesirable because subsequent insults of liquid to the once-swollen composite permeated through the composite at much slower rates and thereby increased the risk of pooling of liquid at the insult point and increased the potential for leaks. The liquid permeability of the pads therefore decreased as the relative amount of gel in the pad increased. Above some critical relative amount of gel, and therefore below some critical porosity, the permeability was inadequate to provide good acquisition rates of subsequent insults. Because the porosity scaled with the relative amount of gel in the absorbent structure according to the equation (20), the critical porosity also depended on the relative amount of gel present in the composite, and could be used to define a critical weight fraction of gel which could be present and still afford the desired porosity, and hence permeability, of the absorbent structure. The critical amount of gel value was transformed into a critical CRC value by calculation using also the design value for the “desired liquid pickup”. The determined critical CRC value was the maximum CRC value for the polymer that could be used to provide the necessary porosity as defined in this invention. For this preferred aspect of the invention, the optimum CRC used for the dryness criterion might not be larger than the critical CRC as defined herein. The design of the absorbent structure (the mass of fluff, the mass of super-absorbent polymer cumulation needed for a design mass of liquid) might be determined by means of equation (28).

Construction of the Absorbent Media: Each super-absorbent polymer was fabricated into a 7.62 cm diameter pad using 1.00 g of super-absorbent polymer (30-50 mesh) and 1.06 g of fluff plus a 7.62 cm diameter tissue on top and bottom. The tissues contributed 0.15 g to the mass of the composite. The 6 percent excess of fluff allowed for the wastage from the “fiber nits” that did not pass through the fiber sieve in the pad former. The super-absorbent polymer was gradually fed into the unit by means of a vibrating feeder, simultaneously with the fluff, which was gradually added by hand through a small slot, so that the pad former acted like a continuous stirred tank reactor for blending the components. A HEPA vacuum cleaner was used to pull the mixture onto the tissue paper. The pad was consolidated by pressing the pad for 45 seconds at 100° C. with 0.318 cm shims in a DAKE brand hydraulic press. Each pad was weighed after pressing and weighed approximately 2.15 grams. After pressing, pads were stored individually in labeled plastic Petri dishes. Subsequent swelling was also done in the Petri dish, in most cases.

Saturation and Blotting Technique: To each pad in its Petri dish was added the amount of saline equal to the CRC of the polymer used in that pad multiplied by the mass of super-absorbent polymer cumulation present. The saline solution was spread evenly over the entire area of the pad so that it was uniformly wet. The dish was covered and polymer swelling was permitted for 60 minutes.

The Petri dish cover was removed momentarily and a stack of four 7.62 cm blotter disks was placed on top of the wet pad. The cover was replaced and the Petri dish was turned over. The bottom of the dish was then removed and another stack of four 7.62 cm blotter disks was placed on top of the wet pad. This maneuver yielded a sandwich structure with the wet pad between two stacks of blotter card. Then a 8.26 cm diameter, 5-kg weight was added on top of the sandwich to compress the blotters against the wet pad. After waiting 5.0 minutes, the 5 kg weight was removed, and the top stack of blotters was carefully lifted off the pad, using a spatula. The lid of the Petri dish was tared, and placed on top of the blotted pad. The dish was again turned over and the other stack of blotters was removed and then the blotted pad was weighed. If the stacks of blotter cards were fully saturated after this procedure (determined by visual observation), a new set of dry blotters was positioned around the pad and the blotting was repeated.

Pad Thickness: The compressibility of wet composites was measured in order to gain information on how the pad volume changed with the swelling and modulus of a super-absorbent polymer. Pads were made as described above, but varying the amount of super-absorbent polymer such that the mass fraction of polymer in the pad varied between 0.12 and 0.7. After consolidating the pad with heat and pressure as described earlier, 0.9 percent NaCl solution was poured onto the pad. The amount of saline solution equaled one times the respective CRC of the polymer. After waiting 60 minutes for liquid absorption, the thickness was measured under loads of 0.02, 0.1, 0.2, 0.3, 0.4 psi. The thickness of the composites was measured with a modified bulk meter from Brown and Sharp North Kingstown, R.I.). After measuring the final thickness value, the pads were blotted according to the procedure described earlier, and the actual swelling extent of the polymer in the pad thereby determined.

Procedure for obtaining FIG. 3: Using the dimensions of the pad and the measured swelling extent of the super-absorbent polymer in the pad, and the masses of each component of the pad, the porosity of the pad was calculated at each loading. A plot of the porosity versus pressure was constructed from the data. The trend of the data was fit using a non-linear least squares procedure to equation (21) and the value of the exponent n was thereby derived for each value of fₛ. A graphical plot of the values of exponent n versus the polymer mass fraction fₛ was then constructed. The trend of the data was obtained by means of a non-linear least squares fitting procedure using the following equation:
n=fₛ^{a}+b
and the obtained values of a=1.83 and b=0.07 used with the equation to compute values of n for pads containing any desired fraction of super-absorbent polymer in their construction.

In further consideration of FIGS. 3 and 4, the porous quality value concept relates to a composite containing super-absorbent polymer. The inventors have discovered that the porosity of the composite depends upon the ratio of the compression applied to the composite to the shear modulus of the swollen gel component, and to the mass fraction of swollen gel in the composite via a mathematical power law. The exponent of the power law reflects the compression sensitivity of the composite to the mass fraction of swollen gel. FIGS. 3 and 4 show the bases for the exponent n as dependent on the mass fraction of swollen gel and also for the exponent value of 0.54 as used in the determination of the optimum CRC for a desired porous quality value in the following two equations:

$$CRC = {F [ {( \frac{1}{{R}_{ϕ}} )}^{\frac{1}{{f}_{s}^{1.83} + 0.07}} - 1 ]}^{0.54}$$

$$and$$

$${m}_{total} = \frac{{m}_{liq}}{\{ ( 1 - {f}_{s} ) {C}_{stranding} + {Ff}_{s} {Φ [ {( \frac{1}{{R}_{ϕ}} )}^{\frac{1}{{f}_{s}^{1.83} + 0.07}} - 1 ]}^{0.54} \}} .$$

In applying the above to a useful application, the porosity ratio R_{φ} must necessarily be quantified. In this regard, and given the benefit of the description of the invention in the foregoing, it is readily appreciated that a desirable range for the porosity ratio R_{φ} is a value between 0.4 and 0.95 insofar that (a) a value greater than 0.95 is associated with a particle that is so essentially rigid that no change in porosity will meaningfully occur with the absorption of liquid and that that (b) a value less than 0.4 is associated with a particle that will most likely swell to block the transfer of liquid flow given the expected fluid flow into the composite and that the necessary relationships for a useful composite will break down. In summary, the porosity ratio R_{φ} requires a value between 0.4 and 0.95 wherein 0.4 denotes an absorbent medium having a minimal porous quality after absorption of said liquid mass and 0.95 denotes an absorbent medium having a maximal porous quality after absorption of said liquid mass. In this regard, porosity ratio R_{φ} is a porous quality value which can be pinpointed to a specific value based upon the judgment of the designer respective to porosity performance in the composite.

With the benefit of the modeled characterization of a composite and the empirically acquired data, there are a number of ways in which the foregoing can be used to design a super-absorbent composite. The following approaches summarize key example solutions to the absorbent composite design challenge.

An optimal amount of super-absorbent polymer cumulation for effectively minimizing free aqueous liquid after a targeted weight of aqueous liquid (such that the super-absorbent polymer cumulation with the targeted weight of absorbed aqueous liquid provides sustained tactile dryness), where the super-absorbent polymer has an affiliated centrifuge retention capacity value, is between a 1.18 and a 2.22 multiple of an absorption design-instance parameter derived from the aqueous liquid weight and from the centrifuge retention capacity value according to

$$K = \frac{{m}_{liq}}{CRC}$$

where
   m_{liq} is a value denoting the targeted weight of liquid,
   CRC is the centrifuge capacity value having units of mass of liquid per mass of dry super-absorbent polymer, and
   K is the absorption design-instance parameter.

When a permeable substruction meshwork of a mass of intertwined stranding (the stranding having an affiliated absorption capacity value) is also a part of the composite, then the above parameter (K) is derived from

$$\underline{K} = {m}_{liq} ( \frac{{m}_{liq} - {C}_{stranding} {m}_{stranding}}{{CRCm}_{liq}} )$$

where
   C_{stranding} is the absorption capacity value having units of mass of liquid per mass of dry stranding, and
   m_{stranding} is a value denoting the mass of stranding.

When the mass of polymer and the dryness quality are defined, then the mass of all the stranding in the above composite can be defined from

$${m}_{stranding} = \frac{{m}_{liq} - Φ ( CRC ) {m}_{sap}}{{C}_{stranding}}$$

where
   m_{stranding} is a value denoting the cumulative mass of all the stranding,
   m_{liq} is a value denoting the predefined mass of liquid to be absorbed,
   Φ is the dryness quality value,
   CRC is the centrifuge retention capacity value having units of mass of liquid per mass of dry super-absorbent polymer,
   mₛₐₚ is a value denoting the cumulative mass of all the super-absorbent polymer particles, and
   C_{stranding} is the absorption capacity value having units of mass of liquid per mass of dry stranding.

When a predefined porous quality is provided (the porous quality denoted by a porous quality value between 0.4 and 0.95 where 0.4 denotes an absorbent medium having a minimal porous quality after absorption of the liquid mass and 0.95 denotes an absorbent medium having a maximal porous quality after absorption of the liquid mass), the centrifuge retention capacity value in the above can be determined according to

$$CRC = {F [ {( \frac{1}{{R}_{ϕ}} )}^{\frac{1}{{f}_{s}^{1.83} + 0.07}} - 1 ]}^{0.54} ,$$

where
F is 40.58 with units of mass of liquid per mass of dry super-absorbent polymer,
R_{φ} is the porous quality value, and
fₛ is a super-absorbent polymer mass fraction value according to

$${f}_{s} = \frac{{m}_{sap}}{{m}_{sap} + {m}_{stranding}} .$$

The polymer mass fraction in the foregoing can be defined specifically or iteratively (against the CRC) either by hand or with the benefit of a computer. A polymer conformant to the CRC can then be used in the composite.

Another approach to using the equations is to define a value denoting a mass of liquid to be absorbed by the medium, define the desired dryness quality value, define the desired porous quality value, define the super-absorbent polymer mass fraction value, select the stranding type and acquire the affiliated absorption capacity value, and calculate the mass of the intermixture of the super-absorbent polymer and stranding components according to

$${m}_{total} = \frac{{m}_{liq}}{\{ ( 1 - {f}_{s} ) {C}_{stranding} + {Ff}_{s} {Φ [ {( \frac{1}{{R}_{ϕ}} )}^{\frac{1}{{f}_{s}^{1.83} + 0.07}} - 1 ]}^{0.54} \}}$$

where
mₜₒₜₐₗ is a value denoting the intermixture mass having units of mass of dry super-absorbent polymer in addition mass of dry stranding,
m_{liq} is the value denoting the mass of liquid to be absorbed,
fₛ is the super-absorbent polymer mass fraction value,
F is 40.58 with units of mass of liquid per mass of dry super-absorbent polymer,
Φ is the dryness quality value,
R_{φ} is the porosity quality value, and
C_{standing} is the absorption capacity value having units of mass of liquid per mass of dry stranding;
   derive the value for the mass of the super-absorbent polymer component according to
   mₛₐₚ=fₛmₜₒₜₐₗ
where mₛₐₚ is the value denoting the super-absorbent polymer component mass;
   derive the value for the mass of the stranding component according to
   m_{stranding}=(1−fₛ)mₜₒₜₐₗ
where m_{stranding} is the stranding component mass value;
and derive the calculated centrifuge capacity value according to

$$CRC = \frac{{m}_{liq} - ( 1 - {f}_{s} ) {C}_{stranding} {m}_{total}}{Φ {f}_{s} {m}_{total}} .$$

The polymer mass fraction in the foregoing can be defined specifically or iteratively (against any or all of the CRC, quality value, and porosity value) either by hand or with the benefit of a computer. A polymer conformant to the CRC can then be used in the composite.

The equations of the invention can be used in various approaches to design. As an example, presuming that the type of super-absorbent polymer and the type of stranding are given, the design process can proceed by the steps of (a) defining a value denoting a mass of liquid to be absorbed by the medium of the composite; (b) defining a value denoting a mass of super-absorbent polymer cumulation to establish a polymer component portion in the medium; (c) defining the dryness quality value; (d) determining a value denoting a mass of the stranding to establish a stranding component portion; (e) calculating the appropriate CRC needed for the super-absorbent; (f) selecting a super-absorbent polymer having a measured centrifuge retention capacity value essentially equivalent to the calculated centrifuge retention capacity value; (g) measuring a quantity of the super-absorbent polymer essentially equivalent to the super-absorbent polymer component mass value to establish a super-absorbent polymer component portion; (h) measuring a quantity of stranding of the stranding type essentially equivalent to the stranding component mass value to establish a stranding component portion; and (i) disposing the super-absorbent polymer component portion throughout the stranding component portion to provide the medium.

In one alternative, the disposing step involves (a) positioning a first tissue cover in a pad former; (b) intermixing the super-absorbent polymer portion and stranding portion to provide the absorption medium; (c) placing the absorption medium upon the first tissue cover; (d) positioning a second tissue cover upon the disposed absorption medium; and (e) heating and compressing the first tissue, the second tissue, and the disposed absorption medium to a predefined thickness.

An alternative approach to design involves use of a computer to conveniently derive values for the absorbent medium. In this situation, the computer has databases holding data pairs of super-absorbent polymer alternatives and stranding alternatives along with their affiliated CRC and absorption capacity information as well as with database space to hold data quantities for the particular case being solved. The computer is programmed to solve the equations of the present invention and to implement the step of receiving, into the computer database, data quantities for (a) the value denoting a mass of liquid to be absorbed by the medium, (b) the dryness quality, (c) the desired porous quality value, (d) a desired super-absorbent polymer mass fraction value, and (e) an absorption capacity value correspondent to a stranding type. After the data has been assimilated from entry into the database by the designer, the computer then proceeds to solve the equations and determine the mass of the intermixture of the super-absorbent polymer component and the stranding component; to derive a value for the mass of the super-absorbent polymer; derive the value for the mass of the stranding component; derive the CRC, and select super-absorbent polymer candidates which have a CRC essentially equivalent to the CRC. The computer then activates the monitor to display the identifier for the super-absorbent polymer, the polymer mass value, and the stranding component mass value. Such a system can be readily deployed in a computerized spreadsheet application or database application having the capability to solve the equations of the foregoing disclosure. In one embodiment, an IBM Personal Computer 300PL using a 400 MHz CPU with a 6 GB Hard Drive from IBM Corporation and a Windows 98 operating system with Excel spreadsheet by Microsoft Corporation provides a platform for the computer-implemented form of the invention. Many different approaches to computer architectural deployment within the context of the above and given the benefit of the above are considered by the applicants to be generally apparent to those of skill.

The present invention and the illustration of the computerized approaches in the present invention can be conveniently modified by those of skill, once given the benefit of this disclosure, to achieve the utility of the present invention without departing from the spirit of the present invention. It should be understood that the description and discussion herein has been presented by way of enabling example and explanation and that the breadth and scope of the present invention should be identified in accordance with the following claims and their equivalents.

1. A method of deriving values for an absorbent medium having super-absorbent polymer dispersed throughout a permeable substruction meshwork of a mass of intertwined stranding, comprising the computer-implemented steps of:
receiving into a computer database data quantities for
a value denoting a mass of liquid to be absorbed by said medium,
a dryness quality value between 0.45 and 0.85 wherein 0.45 denotes an medium having a maximal dryness quality after absorption of said liquid mass and 0.85 denotes an medium having a minimal dryness quality after absorption of said liquid mass,
a porous quality value between 0.4 and 0.95 wherein 0.4 denotes an medium having a minimal porous quality after absorption of said liquid mass and 0.95 denotes an medium having a maximal porous quality after absorption of said liquid mass,
a super-absorbent polymer mass fraction value, and
an absorption capacity value correspondent to a stranding type; and
determining the mass of an intermixture of a super-absorbent polymer component and a stranding component according to
$${m}_{total} = \frac{{m}_{liq}}{\{ ( 1 - {f}_{s} ) {C}_{stranding} + {Ff}_{s} {Φ [ {( \frac{1}{{R}_{ϕ}} )}^{\frac{1}{{f}_{s}^{1.83} + 0.07}} - 1 ]}^{0.54} \}}$$
wherein
mₜₒₜₐₗ is a value denoting said intermixture mass having units of mass of dry super-absorbent polymer in addition with mass of dry stranding,
m_{liq} is said value denoting said mass of liquid to be absorbed,
fₛ is said super-absorbent polymer mass fraction value,
F is 40:58 with units of mass of liquid per mass of dry super-absorbent polymer,
Φ is said dryness quality value,
R_{φ} is said porosity quality value, and
C_{stranding} is said absorption capacity value having units of mass of liquid per mass of dry stranding;
deriving a value for the mass of said super-absorbent polymer component according to
mₛₐₚ=fₛmₜₒₜₐₗ
wherein
mₛₐₚ is said value denoting said super-absorbent polymer component mass;
deriving a value for the mass of said stranding component according to
m_{stranding}=(1−fₛ)mₜₒₜₐₗ
wherein
m_{stranding} is said stranding component mass value;
deriving a calculated centrifuge capacity value according to
$$CRC = \frac{{m}_{liq} - ( 1 - {f}_{s} ) {C}_{stranding} {m}_{total}}{Φ {f}_{s} {m}_{total}}$$
wherein
CRC is said calculated centrifuge capacity value having units of mass of liquid per mass of dry super-absorbent polymer;
selecting a super-absorbent polymer having a measured centrifuge retention capacity value essentially equivalent to said calculated centrifuge retention capacity value; and
displaying upon a monitor of said computer an identifier for said super-absorbent polymer, said polymer mass value, and said stranding component mass value.

2. A method of making an absorbent medium comprising the step of:
intermixing permeable substruction stranding and a mass of super-absorbent polymer particles into a meshwork for absorbing a predefined mass of liquid to a predefined dryness quality, each of said super-absorbent polymer particles having an affiliated centrifuge retention capacity value, said stranding having an affiliated absorption capacity value, said dryness quality denoted by a dryness quality value between 0.45 and 0.85 wherein 0.45 denotes an absorbent medium having a maximal dryness quality after absorption of said liquid mass and 0.85 an absorbent medium having a minimal dryness quality after absorption of said liquid mass, the cumulative mass of all said stranding being
$${m}_{stranding} = \frac{{m}_{liq} - Φ ( CRC ) {m}_{sap}}{{C}_{stranding}}$$
wherein
m_{stranding} is a value denoting said cumulative mass of all said stranding,
m_{liq} is a value denoting said predefined mass of liquid to be absorbed,
Φ is said dryness quality value,
CRC is said centrifuge retention capacity value having units of mass of liquid per mass of dry super-absorbent polymer,
mₛₐₚ is a value denoting the cumulative mass of all said super-absorbent polymer particles, and
C_{stranding} is said absorption capacity value having units of mass of liquid per mass of dry stranding.

3. The method of claim 2 wherein, in said intermixing step, said super-absorbent polymer particles and stranding are intermixed to further achieve a predefined porous quality, said porous quality denoted by a porous quality value between 0.4 and 0.95 wherein 0.4 denotes an absorbent medium having a minimal porous quality after absorption of said liquid mass and 0.95 denotes an absorbent medium having a maximal porous quality after absorption of said liquid mass, and
wherein said centrifuge retention capacity value is determined according to
$$CRC = {F [ {( \frac{1}{{R}_{ϕ}} )}^{\frac{1}{{f}_{s}^{1.83} + 0.07}} ]}^{0.54} ,$$
wherein
F is 40.58 with units of mass of liquid per mass of dry super-absorbent polymer,
R_{φ} is said porous quality value, and
fₛ is a super-absorbent polymer mass fraction value according to
$${f}_{s} = \frac{{m}_{sap}}{{m}_{sap} + {m}_{stranding}} .$$

4. A method of making an absorbent medium having a permeable substruction meshwork of a mass of intertwined stranding, comprising the steps of:
defining a value denoting a mass of liquid to be absorbed by said medium;
defining a value denoting a mass of super-absorbent polymer to establish a polymer component portion in said medium, said polymer having an affiliated centrifuge retention capacity value;
defining a dryness quality value between 0.45 and 0.85 wherein 0.45 a medium having a maximal dryness quality after absorption of said liquid mass and 0.85 denotes a medium having a minimal dryness quality after absorption of said liquid mass;
determining a value denoting a mass of said stranding to establish a stranding component portion, said stranding having an affiliated absorption capacity value, said mass of stranding determined as
$${m}_{stranding} = \frac{{m}_{liq} - Φ ( CRC ) {m}_{sap}}{{C}_{stranding}}$$
wherein
m_{stranding} is said value denoting said mass of stranding,
m_{liq} is said value denoting said mass of liquid to be absorbed,
φ is said dryness quality value,
CRC is said centrifuge retention capacity value having units of mass of liquid per mass of dry super-absorbent polymer,
mₛₐₚ is said mass of super-absorbent polymer, and
C_{stranding} is said absorption capacity value having units of mass of liquid per mass of dry stranding;
measuring a quantity of super-absorbent polymer essentially equivalent to said polymer mass value to establish said super-absorbent polymer component portion;
measuring a quantity of stranding essentially equivalent to said stranding mass value to establish said stranding component portion; and
disposing said super-absorbent polymer component portion throughout said stranding component portion to provide said medium.

5. A method of making an absorbent medium having a permeable substruction meshwork of a mass of intertwined stranding, comprising the steps of:
defining a value denoting a mass of liquid to be absorbed by said medium;
defining a dryness quality value between 0.45 and 0.85 wherein 0.45 denotes a medium having a maximal dryness quality after absorption of said liquid mass and 0.85 denotes a medium having a minimal dryness quality after absorption of said liquid mass;
defining a porous quality value between 0.4 and 0.95 wherein 0.4 denotes a medium having a minimal porous quality after absorption of said liquid mass and 0.95 denotes a medium having a maximal porous quality after absorption of said liquid mass;
defining a super-absorbent polymer mass fraction value;
selecting a stranding type, said stranding type having an affiliated absorption capacity value;
determining the mass of an intermixture of a super-absorbent polymer component and a stranding component according to
$${m}_{total} = \frac{{m}_{liq}}{\{ ( 1 - {f}_{s} ) {C}_{stranding} + {Ff}_{s} {Φ [ {( \frac{1}{{R}_{ϕ}} )}^{\frac{1}{{f}_{s}^{1.83} + 0.07}} - 1 ]}^{0.54} \}}$$
wherein
mₜₒₜₐₗ is a value denoting said intermixture mass having units of mass of dry super-absorbent polymer in addition with mass of dry stranding,
m_{liq} is said value denoting said mass of liquid to be absorbed,
fₛ is said super-absorbent polymer mass fraction value,
Fₛ is 40.58 with units of mass of liquid per mass of dry super-absorbent polymer,
Φ is said dryness quality value,
R_{φ} is said porosity quality value, and
C_{stranding} is said absorption capacity value having units of mass of liquid per mass of dry stranding;
deriving a value for the mass of said super-absorbent polymer component according to
mₛₐₚ=fₛmₜₒₜₐₗ
wherein
mₛₐₚ is said value denoting said super-absorbent polymer component mass;
deriving a value for the mass of said stranding component according to
m_{stranding}=(1−fₛ)mₜₒₜₐₗ
wherein
m_{stranding} is said stranding component mass value;
deriving a calculated centrifuge capacity value according to
$$CRC = \frac{{m}_{liq} - ( 1 - {f}_{s} ) {C}_{stranding} {m}_{total}}{Φ {f}_{s} {m}_{total}}$$
wherein
CRC is said calculated centrifuge capacity value having units of mass of liquid per mass of dry super-absorbent polymer;
selecting a super-absorbent polymer having a measured centrifuge retention capacity value essentially equivalent to said calculated centrifuge retention capacity value;
measuring a quantity of said super-absorbent polymer essentially equivalent to said super-absorbent polymer component mass value to establish a super-absorbent polymer component portion;
measuring a quantity of stranding of said stranding type essentially equivalent to said stranding component mass value to establish a stranding component portion; and
disposing said super-absorbent polymer component portion throughout said stranding component portion to provide said medium.

6. The method of either of claims 4 or 5 wherein said intertwined stranding comprises cellulose Ruff.

7. The method of either of claims 4 or 5 wherein said intertwined stranding comprises a permeable sponge.

8. The method of either of claims 4 or 5 wherein said intertwined stranding comprises a fibrous polymer.

9. The method of either of claims 4 or 5 wherein said disposing step further comprises the steps of:
positioning a first tissue cover in a pad former;
intermixing said super-absorbent polymer portion and stranding portion to provide said absorption medium;
placing said absorption medium upon said first tissue cover;
positioning a second tissue cover upon said disposed absorption medium; and
heating and compressing said first tissue, said second tissue, and said disposed absorption medium to a predefined thickness.

10. Super-absorbent polymer cumulation for absorbing a targeted weight of aqueous liquid, said super-absorbent polymer cumulation having an affiliated centrifuge retention capacity value, said super-absorbent polymer cumulation having a super-absorbent mass between a 1.18 and a 2.22 multiple of an absorption design-instance parameter derived from said aqueous liquid weight and from said centrifuge retention capacity value according to
$$K = \frac{{m}_{liq}}{CRC}$$
wherein
m_{liq} is a value denoting said targeted weight of liquid,
CRC is said centrifuge capacity value having units of mass of liquid per mass of dry super-absorbent polymer, and
K is said absorption design-instance parameter;
so that a sufficiently minimal amount of super-absorbent polymer is provided for effectively minimizing free aqueous liquid within said super-absorbent polymer cumulation after said targeted weight of aqueous liquid has been absorbed such that said super-absorbent polymer cumulation with said targeted weight of absorbed aqueous liquid provides sustained tactile dryness.

11. An absorbent medium for absorbing a targeted weight of aqueous liquid, comprising:
super-absorbent polymer dispersed throughout a permeable substruction meshwork, said permeable substruction meshwork having a mass of intertwined stranding, said stranding having an affiliated absorption capacity value, said super-absorbent polymer having an affiliated centrifuge retention capacity value, said super-absorbent polymer having a super-absorbent mass between a 1.18 and a 2.22 multiple of an absorption design-instance parameter derived from said aqueous liquid weight, said absorption capacity value, said mass of stranding, and said centrifuge retention capacity value according to
$$K = {m}_{liq} ( \frac{{m}_{liq} - {C}_{stranding} {m}_{stranding}}{{CRCm}_{liq}} )$$
wherein
m_{liq} is a value denoting said targeted weight of liquid,
C_{stranding} is said absorption capacity value having units of mass of liquid per mass of dry stranding,
CRC is said centrifuge capacity value having units of mass of liquid per mass of dry super-absorbent polymer,
m_{stranding} is a value denoting said mass of stranding, and
K is said absorption design-instance parameter;
so that a sufficiently minimal amount of super-absorbent polymer is provided for effectively minimizing free aqueous liquid within said absorbent medium after said targeted weight of aqueous liquid has been absorbed such that said absorbent medium with said targeted weight of absorbed aqueous liquid provides sustained tactile dryness.

12. The medium of claim 11 wherein said intertwined stranding comprises cellulose fluff.

13. The medium of claim 11 wherein said intertwined stranding comprises a permeable sponge.

14. The medium of claim 11 wherein said intertwined stranding comprises a fibrous polymer.
